(19) United States
(12) Patent Application Publication
(10) Pub. No.: US 2026/0284423 A1
(43) Pub. Date: Sep. 24, 2026

(54) SKIN CARE DEVICE

(71) Applicant: Triple A Finance GmbH & Co. KG, Bielefeld (DE)

(72) Inventor: Patric Moammer, New York, NY (US)

(21) Appl. No.: 18/881,229

(22) PCT Filed: Jul. 6, 2023
(86) PCT No.: PCT/EP2023/068750

§ 371 Date: Jan. 3, 2025

(30) Foreign Application Priority Data
Jul. 6, 2022 (DE) 102022206881.5

(51) Int. Cl. A61N 5/06; A61N 5/00

(52) U.S. Cl. CPC A61N 5/0616; A61N 2005/007

(57) ABSTRACT

A skin care device is described with a portable base body (2) which has a skin care surface (4) and, outside the skin care surface (4), a receiving section (8), with a holding element (6) which is designed to be arranged on the receiving portion (8) of the base body (2) and thereby to bring a portion of a skin care element (12) to be arranged at least on a region of the skin care surface (4) into clamping engagement between itself and the receiving portion (8), and with locking means (10a, 10b) for releasably fastening the holding element (6) to the receiving portion (8) of the base body (2). The special feature of the invention is that the locking means (10a, 10b) are designed to bring the holding element (6) into contact with the receiving section (8) of the base body (2), at least in sections, by means of magnetic forces.

The invention relates to a skin care device having a portable base body which has a skin care surface and, outside the skin care surface, a receiving section, having a holding element which is designed to be arranged on the receiving section of the base body and thereby bring a section of a skin care element to be arranged on at least one region of the skin care surface into clamping engagement between itself and the receiving section, and having locking means for releasably fastening the holding element to the receiving section of the base body.

Such a skin care device is known from the prior art, for which reference is made, for example, to EP 2 382 893 A1 or WO 2007/015729 A1 . In the prior art, the holding element for fixing a skin care element, which is usually a textile substrate impregnated with a skin care agent, is brought into mechanical clamping engagement with the receiving section on the base body on the skin care surface. For this purpose, the locking means are designed as mechanical locking means and usually have at least one locking projection or a locking lug, which engages in a locking groove or locking opening formed in the receiving section of the base body. This type of fastening of the holding element has several disadvantages. Firstly, the holding element is difficult to release against the locking force, which cannot be precisely dimensioned, making handling cumbersome. Secondly, it is generally un-avoidable that the skin care element to be arranged on the base body, which is usually designed as a textile substrate, comes to rest between the locking protrusions on the one hand and the locking openings on the other, thereby preventing the locking protrusions from engaging in the locking openings. This leads to com-plications when releasing the holding element from the receiving section of the base body, as the holding element can only be removed from the base body under difficult conditions, since the additional engagement with the skin care element in-advertently increases the locking forces. To avoid these disadvantages, it would be conceivable to remove the skin care element at the points where it comes into contact with the locking protrusions or latching lugs. However, this requires specially manufactured skin care elements on the one hand and complicated handling on the other, as it must always be ensured that the recesses are aligned with the locking protrusions or latching lugs when the skin care element is placed on the skin.

It is an object of the present invention to propose an improved solution for the locking means which avoids the aforementioned problems.

According to the invention, this task is solved with a skin care device having a portable base body which has a skin care surface and, outside the skin care surface, a receiving section, having a holding element which is designed to be arranged on the receiving section of the base body and, in the process, to bring a section of a skin care element to be arranged at least on one region of the skin care surface into clamping engagement between itself and the receiving section, and with locking means for releasably securing the holding element to the receiving portion of the base body, characterized in that the locking means are designed to bring the holding element into contacting engagement with the receiving portion of the base body, at least in sections, by means of magnetic forces.

The special feature of the invention is thus that the locking means are designed to bring the holding element into contact with the receiving section of the base body, at least in sections, by means of magnetic forces. Several advantages can be achieved with this measure according to the invention. The magnetic forces can be dimensioned in such a way that, on the one hand, a sufficient attraction force can be achieved for reliable clamping engagement and, on the other hand, the holding element can be easily released from the receiving section of the base body without excessive force. Furthermore, the solution according to the invention offers the possibility of a flat contact of the holding element on the receiving section while avoiding protrusions, latching lugs or the like. A further advantage resulting from the realization of a flat contact of the holding element on the receiving section is that the skin care element can be easily arranged with a section, in particular an edge section, between the holding element and the receiving section, without impairing the fastening effect of the holding element. Furthermore, the skin care element does not need to be adapted accordingly, but any skin care element can be used, in any arrangement and orientation.

Preferred embodiments and further embodiments of the invention are given in the dependent claims.

Preferably, the holding element is designed to bring a substantially flat skin care element into clamping engagement between itself and the receiving section of the base body, which is designed as a flexible, preferably textile-like, substrate suitable for absorbing liquid and/or viscous care products.

Expediently, the locking means comprise first magnetic means provided on and/or in the holding element and second magnetic means provided on and/or in the receiving section of the base body, the first magnetic means and the second magnetic means being designed and aligned with respect to one another in such a way that an attractive force acting on the first magnetic means and the second magnetic means is generated, wherein in particular (1.) the first magnet means comprise magnetic material and the second magnet means comprise magnetizable material or (2.) the first magnet means comprise magnetizable material and the second magnet means comprise magnetic material or (3.) both magnet means each comprise magnetic material.

For maximum utilization of the skin care surface, it should preferably be adjacent to the holding section that receives the holding element.

For an equally optimal utilization of the skin care surface on the one hand and for a particularly effective attachment of the holding element to the receiving section on the other hand, the receiving section on the base body should preferably enclose the skin care surface, for which purpose the holding element in particular can be designed as a circumferential frame and provided to enclose the skin care surface when attached to the receiving section of the base body.

In another preferred embodiment, the receiving section on the base body forms a recess relative to the skin care surface. In particular, the recess serves as a kind of stop for the holding element in order to achieve an essentially stationary alignment and arrangement of the holding element relative to the skin care surface.

Expediently, the receiving section should be essentially strip-shaped.

A further embodiment of the invention is characterized in that the base body is provided with means for heating and/or cooling the skin care surface and/or with means for subjecting the base body and in particular the skin care surface to vibrations and/or with means for generating light radiation.

In the following, a preferred embodiment of the invention is explained in more detail with reference to the attached figures. It shows:

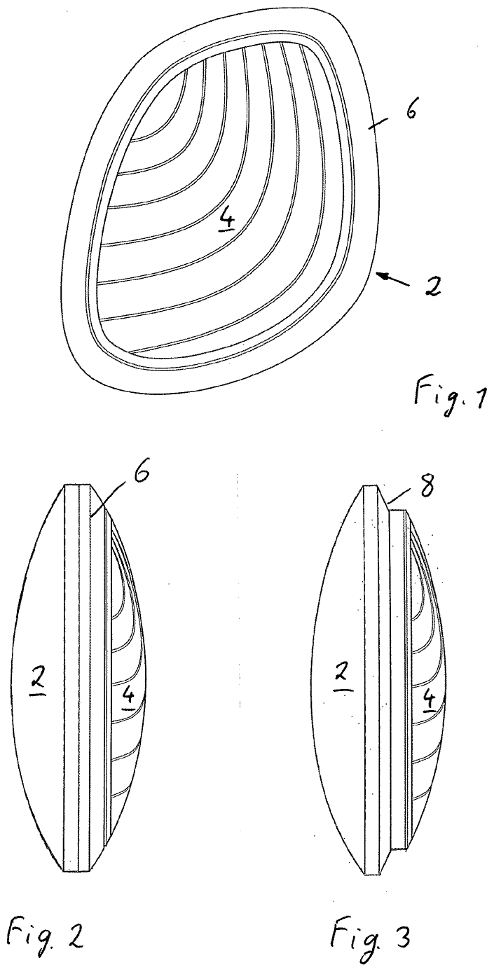

FIG. 1a top view of a skin care device according to a preferred embodiment example with a strip-shaped holding frame arranged on the portable base body;

FIG. 2 the skin care device of FIG. 1 in a side view;

FIG. 3 the skin care device of FIG. 1 in a side view with the holding frame removed;

FIG. 4a a top view of the circumferential holding frame as a single view;

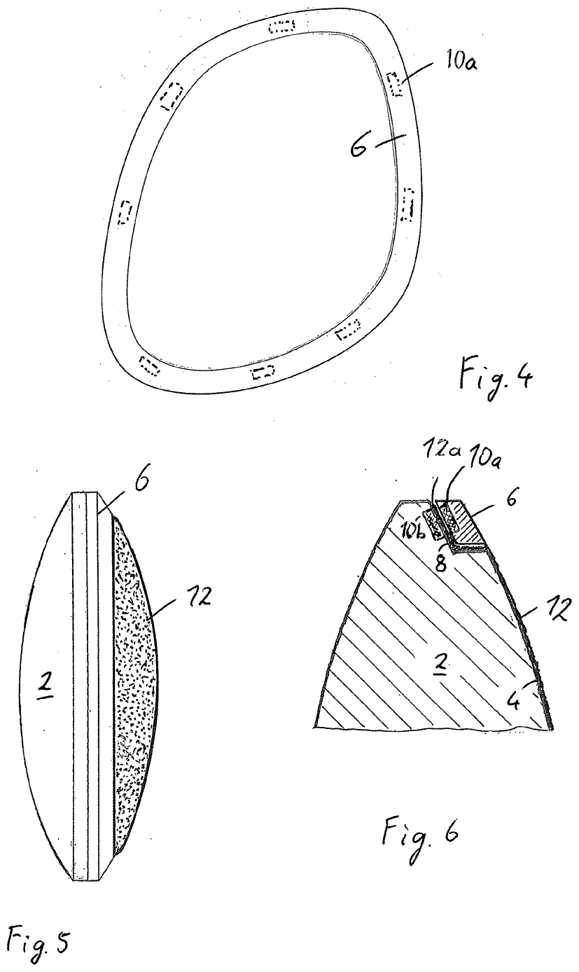

FIG. 5 the same view as FIG. 2, but additionally with a skin care element attached to the skin care device; and

FIG. 6 a cross-section of an edge section of the skin care device of FIG. 1.

FIG. 1 shows a top view of the skin care device according to a preferred embodiment with a portable base body 2, on one side of which a skin care surface 4 is provided. The base body 2 preferably contains a device for heating and/or cooling the skin care surface 4 and/or a device for applying vibrations to the base body 2 and in particular to the skin care surface 4 and/or a device for generating light radiation for light therapy, the said devices not being shown in the figures.

Furthermore, FIG. 1 shows a holding frame 6 arranged on the base body 2, which is strip-shaped in the illustrated embodiment example and encloses the skin care surface 4.

While FIG. 2 shows the skin care device of FIG. 1 with the holding frame 6 attached to it, FIG. 3 shows the skin care device from the same perspective with the holding frame 6 removed and thus reveals a receiving section 8 which is formed on the base body 2 as a recess relative to the skin care surface 4. The receiving section 8 also has a strip shape, which is essentially adapted to the strip shape of the holding frame 6, and encloses the skin care surface 4. The circumferential receiving section 8 receives the holding frame 6, which is also circumferential, and is thus arranged accordingly on the receiving section 8. In the illustrated embodiment, the circumferential holding frame 6 and the circumferential receiving section 8 are dimensioned such that, when arranged on the receiving section 8, the holding frame 6 is substantially aligned with its surface with the skin care surface 4, as FIG. 2 shows. The recess formed by the receiving section 8 relative to the skin care surface 4 in the illustrated embodiment takes up the function of a stop in order to align and arrange the holding frame 6 on the base body 2 without significant tolerances relative to the skin care surface 4. As FIGS. 1 to 3 also show, the receiving section 8 runs along the side edge of the base body 2 and therefore also forms a peripheral edge section of the base body 2 and therefore also of the skin care device.

The holding frame 6 can be detachably attached to the holding section 8 of the base body 2. For this purpose, locking means are provided which have first magnetic elements 10a and second magnetic elements 10b, wherein the first magnetic elements 10a have an opposite magnetic polarity to the second magnetic elements 10b. In the illustrated embodiment according to FIGS. 4 and 6, the first magnetic elements 10a are arranged within the circumferential holding frame 6, as shown in dashed lines in the illustration of FIG. 4, while the second magnetic elements 10b are arranged within the base body 2 essentially directly below the receiving section 8, as FIG. 6 shows. In the illustrated embodiment, the first magnetic elements 10a and the second magnetic elements 10b are each provided as discrete bodies which are spaced apart from one another, preferably substantially equidistantly, and are positioned in such a way that, when the holding frame 6 is arranged on the receiving section 8, a first magnetic element 10a comes to lie adjacent to or at the level of a second magnetic element 10b, as indicated in FIG. 6. The first magnetic elements 10a can have magnetic material and the second magnetic elements 10b can have magnetizable material, or conversely, the first magnetic elements 10a can have magnetizable material and the second magnetic elements 10b can have magnetic material, or alternatively it is also conceivable that the first and second magnetic elements 10a, 10b each have magnetic material. Alternatively, however, it is also conceivable, for example, to form at least one of the two magnetic elements 10a, 10b as a strip-shaped body surrounding the holding frame 6 or the holding section 8. A further alternative possibility could, for example, be to realize at least one of the first or second magnetic elements 10a, 10b by inserting or applying magnetic or magnetizable material, either at discrete points or essentially continuously along the circumferential holding frame 6 or the circumferential receiving section 8 in the form of a strip.

As can also be seen in FIGS. 5 and 6, the holding frame 6 serves to bring a section of a skin care element 12 to be arranged on the skin care surface 4 into clamping engagement between itself and the receiving section 8 when removably attached to the receiving section 8. The skin care element 12 to be used is preferably a flexible, preferably textile-like, substrate suitable for holding liquid and/or viscous care products.

1. Skin care device
with a portable base body (2), which has a skin care surface (4) and a receiving section (8) outside the skin care surface (4),
with a holding element (6) which is designed to be arranged on the receiving portion (8) of the base body (2) and thereby to bring a portion of a skin care element (12) to be arranged at least on a region of the skin care surface (4) into clamping engagement between itself and the receiving portion (8), and
with locking means (10a, 10b) for releasably fastening the holding element (6) to the receiving section (8) of the base body (2),
characterized in that
the locking means (10a, 10b) are designed to bring the holding element (6) into contact with the receiving section (8) of the base body (2), at least in sections, by means of magnetic forces.

2. Skin care device according to claim 1, characterized in that the holding element (6) is designed to bring into clamping engagement between itself and the receiving portion (8) of the base body (2) a substantially flat skin care element (12) which is designed as a flexible, preferably textile-like, substrate suitable for absorbing liquid and/or viscous care products.

3. Skin care device according to claim 1 or 2, characterized in that the locking means comprise first magnet means (10a) provided on and/or in the holding element (6) and second magnet means (10b) provided on and/or in the receiving portion (8) of the base body (2), the first magnet means (10a) and the second magnet means (10b) being designed and aligned with respect to one another in such a way that an attractive force acting on the first magnet means (10a) and the second magnet means (10b) is generated.

4. Skin care device according to claim 3, characterized in that (1.) the first magnet means (10a) comprise magnetic material and the second magnet means (10b) comprise magnetizable material or (2.) the first magnet means (10a) comprise magnetizable material and the second magnet means (10b) comprise magnetic material or (3.) both magnet means (10a, 10b) each comprise magnetic material.

5. Skin care device according to at least one of the preceding claims, characterized in that the receiving section (8) is adjacent to the skin care surface (4).

6. Skin care device according to at least one of the preceding claims, characterized in that the receiving section (8) on the base body (2) encloses the skin care surface (4).

7. Skin care device according to claim 6, characterized in that the holding element (6) is designed as a circumferential frame and is provided to enclose the skin care surface (4) when attached to the receiving section (8) of the base body (2).

8. Skin care device according to at least one of the preceding claims, characterized in that the receiving section (8) on the base body (2) forms a recess relative to the skin care surface (4).

9. Skin care device according to at least one of the preceding claims, characterized in that the receiving section (8) is substantially strip-shaped.

10. Skin care device according to at least one of the preceding claims, characterized in that the base body (2) is provided with means for heating and/or cooling the skin care surface (4) and/or with means for applying vibrations to the base body (2) and in particular to the skin care surface (4) and/or with means for generating light radiation.
